(19) United States
(12) Patent Application Publication
(10) Pub. No.: US 2003/0055683 A1
(43) Pub. Date: Mar. 20, 2003

(54) System and method for disseminating drug information

(76) Inventors: Jerry Tyrone Gibson, Auburn, AL (US); Rhett Dorsey Gibson, Auburn, AL (US); Devin Roy Gibson, Auburn, AL (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/247,237

(22) Filed: Sep. 19, 2002

Related U.S. Application Data
(60) Provisional application No. 60/323,496, filed on Sep. 19, 2001.

(51) Int. Cl. G06F 17/60; G06F 19/00; G01N 33/48; G01N 33/50

(52) U.S. Cl. 705/2; 702/19

(57) ABSTRACT

The present invention provides a system and method for disseminating drug information. In architecture, the system includes a server device containing original drug information. The server device further comprises an update mechanism that creates update drug data for addition to the original drug information, and a transmit mechanism that transmits the update drug data to the remote device upon receiving a request from a remote device for the update drug data. The present invention can also be viewed as a method for disseminating drug information. The method operates by (1) creating update drug data for addition to original drug information; (2) waiting for the remote device to request update drug data; and (3) transmitting the update drug data to the remote device.

### CROSS-REFERENCE TO RELATED APPLICATION

This application claims priority to copending U.S. provisional application entitled, “AUTOMATIC UPDATING DEVICES, METHODS, AND SYSTEMS,” having ser. No. 60,323,496; filed oil Sep. 19, 2001, which is entirely incorporated herein by reference.

### FIELD OF THE INVENTION

The present invention relates to a method and system for updating files, and more particularly, relates to a method and system for disseminating drug information.

### BACKGROUND OF THE INVENTION

Currently, the Food and Drug Administration (FDA) requires that drug manufacturers provide extensive information with each FDA approved drug sold in the United States. This full disclosure of prescribing information must be in a specific format defined by the FDA. The FDA further specifies the content of the information to be included.

Although the FDA specifies the content and format of the information to be included with each approved drug, the FDA gives the drug manufacturer leeway in the exact form of presentation of the information as long as the required information is disseminated with the drug. To accomplish this, the manufacturer provides the required information at a level understandable to the average prescriber and provider of the drug in a written format often including graphs, charts, and chemical formula diagrams. The information is available directly from the FDA, is provided via advertising or promotional materials produced by the drug manufacturer, and is often compiled by third-party organizations that make the compiled information commercially available.

However, this method of presentation is of limited usefulness as the information is often not the most recent version by the time it reaches the physician or pharmacist. Even if the physician or pharmacist seeks information on prescription drugs via a third-party compilation of labeling published on a periodic basis, the information could be as much as a year out of date for established products and could fail to include information on new products. Company-produced materials are generally more current; however, even they may contain information that has been changed since the date of their publication.

Thus, a heretofore unaddressed need exists in the industry to address the aforementioned deficiencies in providing current full disclosure prescribing drug information to physicians and pharmacists in a quick and efficient manner that ensures that the information is always up to date.

### SUMMARY OF THE INVENTION

The present invention provides a system and method for disseminating drug information. In architecture, the system includes a server device containing original drug information. The server device further comprises an update mechanism that creates update drug data for addition to the original drug information, and a transmit mechanism that transmits the update drug data to the remote device upon receiving a request from a remote device for the update drug data.

The invention may also be conceptualized as a method for disseminating drug information. The method operates by (1) creating update drug data for addition to original drug information; (2) waiting for the remote device to request update drug data; and (3) transmitting the update drug data to the remote device.

### BRIEF DESCRIPTION OF THE DRAWINGS

The present invention, as defined in the claims, can be better understood with reference to the following drawings. The components within the drawings are not necessarily to scale relative to each other, emphasis instead being placed upon clearly illustrating the principles of the present invention.

FIG. 1 is a block diagram illustrating the network environment in which a computing device exists including the prescription drug information dissemination system of the present invention.

FIG. 2A is a block diagram illustrating an example of a server device utilizing the server prescription drug information dissemination system of the present invention.

FIG. 2B is a block diagram illustrating an example of a client device utilizing the client prescription drug information dissemination system of the present invention.

FIG. 3 is a flow chart illustrating an example of server prescription drug information dissemination system of the present invention, as shown in FIG. 2A.

FIG. 4 is a flow chart illustrating an example of the server update process, as shown in FIGS. 2A and 3, operating with the server prescription drug information dissemination system of the present invention.

FIG. 5 is a flow chart illustrating an example of the client update process, as shown in FIGS. 2B and 3, operating with the server prescription drug information dissemination system of the present invention.

FIG. 6 is a flow chart illustrating an example of the call center fix process, as shown in FIGS. 2A and 3, operating with the server prescription drug information dissemination system of the present invention.

FIG. 7 is a flow chart illustrating an example of the client prescription drug information dissemination system, as shown in FIG. 2B.

FIG. 8 is a flow chart illustrating an example of the update process, as shown in FIGS. 2B and 7, operating with the client prescription drug information dissemination system of the present invention.

FIG. 9 is a flow chart illustrating an example of the integrity verification process, as shown in FIG. 8, operating with the update process within the client prescription drug information dissemination system of the present invention.

FIG. 10 is a flow chart illustrating an example of a query process, as shown in FIGS. 2A and 7, operating with the client prescription drug information dissemination system of the present invention.

### DETAILED DESCRIPTION OF THE INVENTION

The invention to be described hereafter is for access to and the dissemination of drug information. The preferred embodiment of the present invention comprises a system and method for parsing FDA approved prescription drug package insert information into a database for dissemination to pharmacists and doctors and hospitals and the like to maintain the most up-to-date FDA required prescription drug package insert information. Prescription drug package insert information also refers to prescription drug labeling information and full disclosure package prescribing information.

In alternative embodiments, the prescription drug package insert information dissemination system can disseminate other types of information as well, including but not limited to, FDA drug alerts, Material Safety Data Sheets (MSDS) designed to provide both workers and emergency personnel with the proper procedures for handling or working with a particular substance, operating system updates, and the like.

The prescription drug package insert information dissemination system of the current invention utilizes the capability of client systems to connect to the server prescription drug package insert information dissemination system at will. Thus in the preferred embodiment, all clients connect to the host on a predetermined schedule to get updates to their resident database. Thus, the server never initiates a call to a client system. Furthermore, the client systems will provide notification to a user as to the success or failure of the update to enable a user to easily see when the last update occurred.

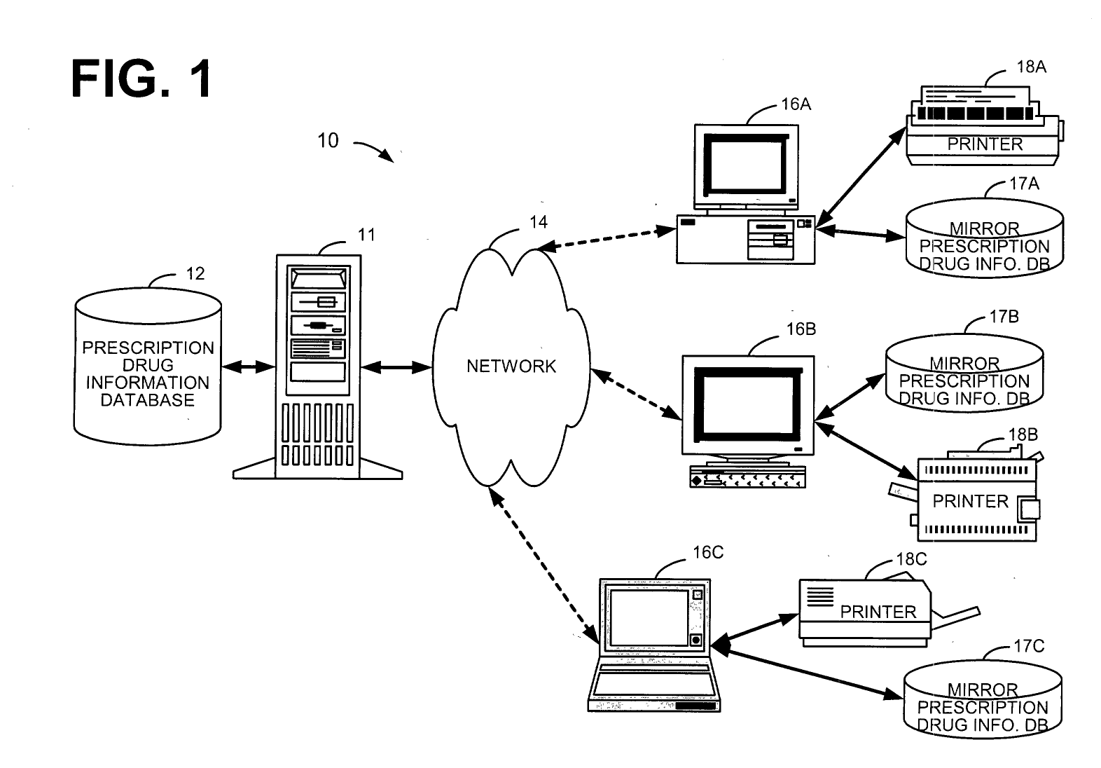

Referring now to the drawings, in which like numerals illustrate like elements throughout the several views, FIG. 1 illustrates the basic components of an intermittent connected prescription drug information dissemination system (PRID) 10 used in connection with the preferred embodiment of the present invention. The PRID system 10 includes client systems 16a, 16b, and 16c. Each client has applications and a local database 17a, 17b, and 17c. A computer server 11 contains applications and a server DB 12 that are accessed by client systems 16(a-c) via intermittent connections, respectively, over network 14. The server 11 runs administrative software for a computer network and provides access to part or all of the network and its devices. The client systems 16(a-c) access the data on computer server 11 and may provide over a network 14, such as but not limited to: the Internet, a local area network (LAN), a wide area network (WAN), or public switched telephone network (PSTN) via a telephone line using a modem or other like networks.

The structure and operation of the PRID system 10 enables the server 11 and the server database 12 associated therewith to handle clients more efficiently than previously known systems. Particularly, the present invention provides a manner of organizing data of the server database into updates that enable a remote client system to update its remote file more efficiently. Periodically, an update file is created for each client with all relevant changes since the last modification of the client database. When the clients systems 16(a-c) connect to the server 11, the modification files associated with the client are transmitted to the client to be used for updating each client's individual database.

The client systems 16(a-c) may each be located at remote sites. Thus, when a user at one of the remote client systems 16(a-c) desires to be updated with the current information from the shared database at the server 11, the client system 16(a-c) communicates over the network 14, such as but not limited to WAN, internet, or PSTN lines to access the server 11. Advantageously, the present invention provides a system and method for updating client systems to most efficiently transfer update data on the server 11. Periodically, each client connects to the server and requests the update data for the client. The server creates and transmits update files for update of the client database.

Hence, the present invention provides for a more efficient approach to maintaining synchronization of remote client files. In this approach, the server 11, performs a server update process that accesses one or more other data bases to acquire data changes that must be disseminated to the client databases. These databases accessed by the server include, but are not limited to, any FDA, Material Safety Data Sheet, or operating system management or the like databases to identify updated information. After acquiring the updated information, the server 11 then determines if there is any room for any potential operating system update. If it is determined that there is no room for the any operating system update then the server 11 then removes any OS update information from the database update data to be disseminated. The server separates the database update data into appropriate size units for easy dissemination and prepares a confirmation information for the units so that the clients can verify that all of the database updated data was applied correctly. The server then prepares these units for transmission to the client and may compress data as required.

After the updated data is prepared, the server 11 then disseminates the database update data on demand by the clients. During a connection to a client requesting updates, the server determines if there are problems with the download of data to the client. If there are problems, then the server 11 attempts to provide the opportunity to correct the problem preventing dissemination of the database update data.

The client prescription drug information dissemination system provides a similar processing by scheduling on a predetermined basis the execution of the update process. The update process for the client entails connecting to the server and downloading any new or updated data from the server. Any data downloaded includes a check sum in order for the client to validate that the updated data was properly propagated to the mirror prescription drug database. After downloading the client may decompress any data required and then provides the update data to the mirror prescription information database. After updating the mirror prescription drug database, the client performs the integrity verification by comparing the check sum received from the server with that generated on the client from the,data updated. If it is determined that a particular update did not occur correctly, the client device will attempt to download the data for a predetermined number of times. Upon unsuccessful attempts of downloading the data, the server will then terminate update process. Once the data is downloaded, the client will be alerted in order to inform the user of particular occurrences or demands with regard to the update downloaded from the server 11. These alerts include, but are not limited to, the databases selected by the user for visual updates, the display list of alerts for the selected databases, the alerts selected by the user most recently, system displays of text of the alerts provided and the logs for reporting which alerts were reviewed.

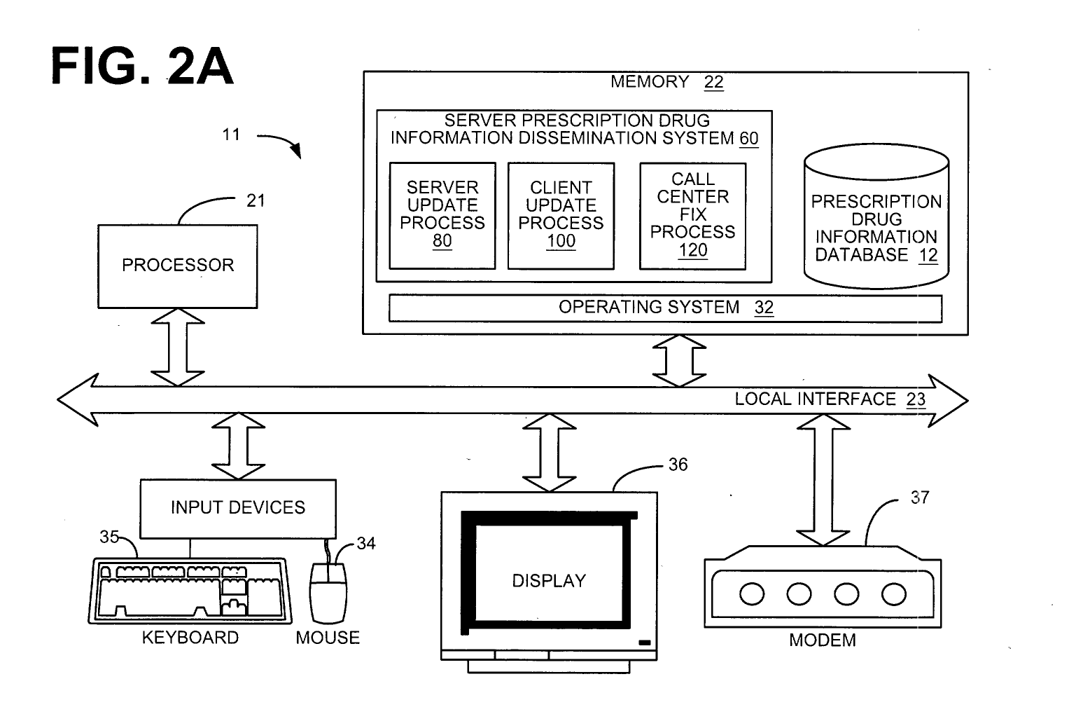

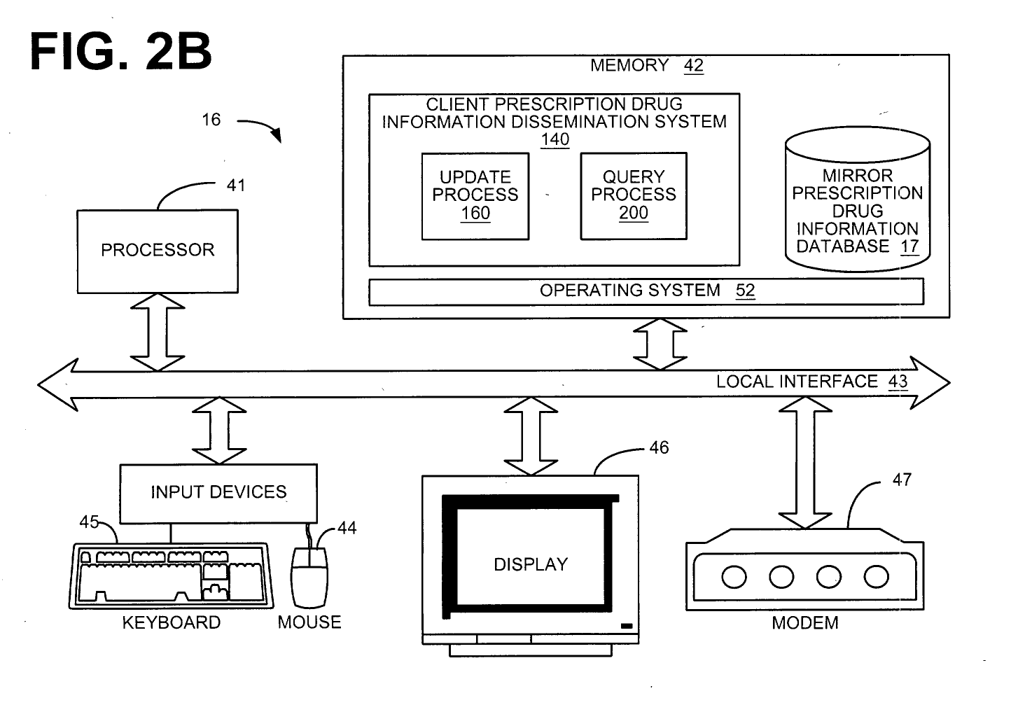

Generally, in terms of hardware architecture, as shown in FIG. 2A, the server 11 include a processor 21, storage memory 22, and one or more input and/or output (I/O) devices (or peripherals) that are communicatively coupled via a local interface 23. The local interface 23 can be, for example but not limited to, one or more buses or other wired or wireless connections, as is known in the art. The local interface 23 may have additional elements, which are omitted for simplicity, such as controllers, buffers (caches), drivers, repeaters, and receivers, to enable communications. Further, the local interface 23 may include address, control, and/or data connections to enable appropriate communications among the aforementioned components.

The processor 21 is a hardware device for executing software that can be stored in memory 22. The processor 21 can be virtually any custom made or commercially available processor, a central processing unit (CPU) or an auxiliary processor among several processors associated with the server 11, and a semiconductor based microprocessor (in the form of a microchip) or a macroprocessor. Examples of suitable commercially available microprocessors are as follows: an 80x86 or Pentium series microprocessor from Intel Corporation, U.S.A., a PowerPC microprocessor from IBM, U.S.A., a Sparc microprocessor from Sun Microsystems, Inc, a PA-RISC series microprocessor from Hewlett-Packard Company, U.S.A., or a 68xxx series microprocessor from Motorola Corporation, U.S.A.

The memory 22 can include any one or combination of volatile memory elements (e.g., random access memory (RAM), such as dynamic random access memory (DRAM), static random access memory (SRAM), etc.)) and nonvolatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 22 may incorporate electronic, magnetic, optical, and/or other types of storage media. Note that the memory 22 can have a distributed architecture, where various components are situated remote from one another, but can be accessed by the processor 21. The new prescription drug information database 12 also resides in memory 22.

The software in memory 22 may include one or more separate programs, each of which comprises an ordered listing of executable instructions for implementing logical functions. In the example of FIG. 2A, the software in the memory 22 includes a suitable operating system (O/S) 32 and the server prescription drug information dissemination system 60 of the present invention. The server prescription drug information dissemination system 60 of the present invention includes a server update process 80, client update process 100 and call center fix process 120.

A non-exhaustive list of examples of suitable commercially available operating systems 32 is as follows: a Windows operating system from Microsoft Corporation, U.S.A., a Netware operating system available from Novell, Inc., U.S.A., an operating system available from IBM, Inc., U.S.A., any LINUX operating system available from many vendors or a UNIX operating system, which is available for purchase from many vendors, such as Hewlett-Packard Company, U.S.A., Sun Microsystems, Inc. and AT&T Corporation, U.S.A. The operating system 32 essentially controls the execution of other computer programs, such as the server prescription drug information dissemination system 60, and provides scheduling, input-output control, file and data management, memory management, and communication control and related services. However, it is contemplated by the inventors that the server prescription drug information dissemination system 60 is applicable on all other commercially available operating systems.

The server prescription drug information dissemination system 60 may be a source program, executable program (object code), script, or any other entity comprising a set of instructions to be performed. When a source program, then the program is usually translated via a compiler, assembler, interpreter, or the like, which may or may not be included within the memory 22, so as to operate properly in connection with the O/S 32. Furthermore, the server prescription drug information dissemination system 60 can be written as (a) an object oriented programming language, which has classes of data and methods, or (b) a procedure programming language, which has routines, subroutines, and or functions, for example but not limited to, C, C++, Pascal, BASIC, FORTRAN, COBOL, Perl, Java, and Ada.

The I/O devices may include input devices, for example but not limited to, a keyboard 25, mouse 24, scanner (not shown), microphone (not shown), etc. Furthermore, the I/O devices may also include output devices, for example but not limited to, a printer (not shown), display 36, etc. Finally, the I/O devices may further include devices that communicate both inputs and outputs, for instance but not limited to, a network interface card (NIC) (not shown) or modulator/demodulator (modem) 27 (for accessing other files, devices, systems, or a network), a radio frequency (PF) or other transceiver (not shown), a telephonic interface (not shown), a bridge (not shown), a router (not shown), etc.

If the server 11 is a PC, workstation, or the like, the software in the memory 22 may further include a basic input output system (BIOS) (omitted for simplicity). The BIOS is a set of essential software routines that initialize and test hardware at startup, start the O/S 32, and support the transfer of data among the hardware devices. The BIOS is stored in ROM so that the BIOS can be executed when the server 11 is activated.

When the server 11 is in operation, the processor 21 is configured to execute software stored within the memory 22, to communicate data to and from the memory 22, and to generally control operations of the server 11 pursuant to the software. The server prescription drug information dissemination system 60 and the O/S 32 are read, in whole or in part, by the processor 21, perhaps buffered within the processor 21, and then executed.

When the server prescription drug information dissemination system 60 is implemented in software, as is shown in FIG. 2A, it should be noted that the server prescription drug information dissemination system 60 can be stored on virtually any computer readable medium for use by or in connection with any computer related system or method. In the context of this document, a computer readable medium is an electronic, magnetic, optical, or other physical device or means that can contain or store a computer program for use by or in connection with a computer related system or method. The server prescription drug information dissemination system 60 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

In an alternative embodiment, where implemented in hardware, the server prescription drug information dissemination system 60 can be implemented with any one or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

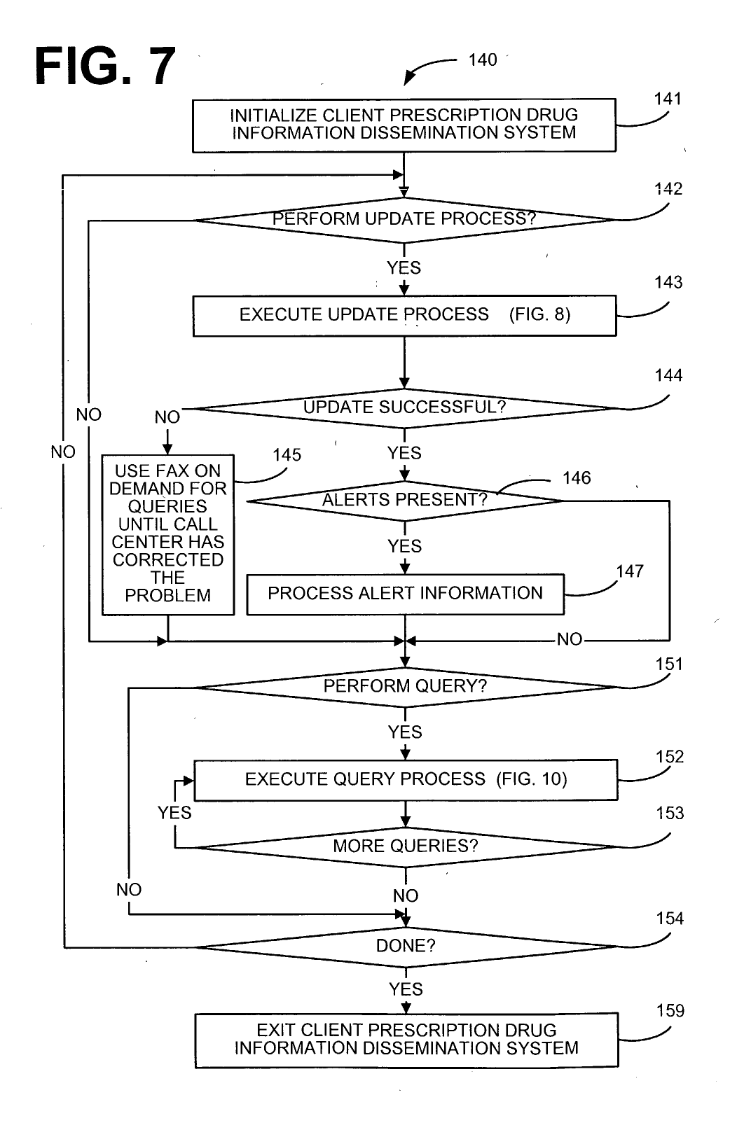

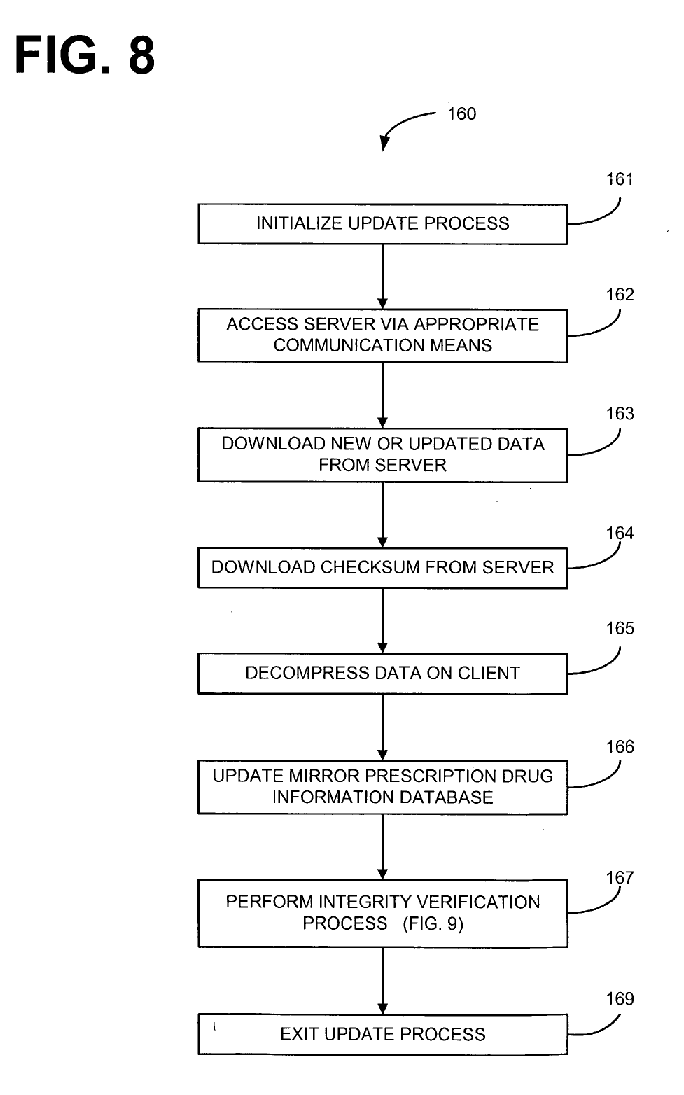

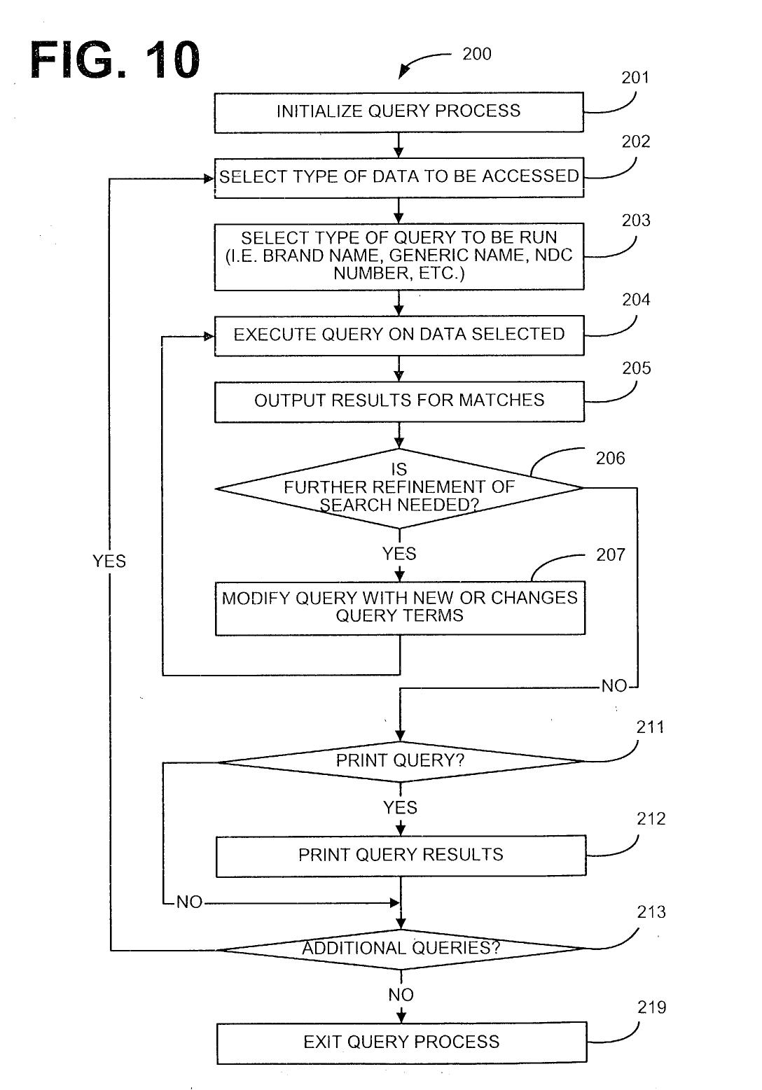

Illustrated in FIG. 2B is the client system 16. The client system 16 includes the client prescription drug information dissemination system 140 which further includes an update process 160 and query process 200 as well as the mirror prescription information database 17 within the computer readable medium such as memory 42. The architecture of client 16 is similar to that of the server 21. The functionality of the processor 41, memory 42, mouse 44, keyboard 45, display 46 and modem 47 are essentially the same as the corresponding items in FIG. 2A described above. As discussed previously, the client prescription drug information dissemination system 140 requests periodic updates on a predetermined schedule from a server 11. These updates are then applied to the mirror prescription drug information database 17, so that the clients mirror prescription drug information database 17 is a mirror of the server prescription drug information database 12. The update process 160 within the client prescription drug information dissemination system 140 is the process for requesting and applying the updates to the mirror prescription drug information database 17. The query process 200 enables a user to access the mirror prescription drug information database 17 on an as requested basis. The client prescription drug information dissemination system 140 is herein described in further detail with regard to FIG. 7. The update process 160 is herein defined and further detailed with regard to FIG. 8 and a query process 200 is herein defined in further detail with regard to FIG. 10.

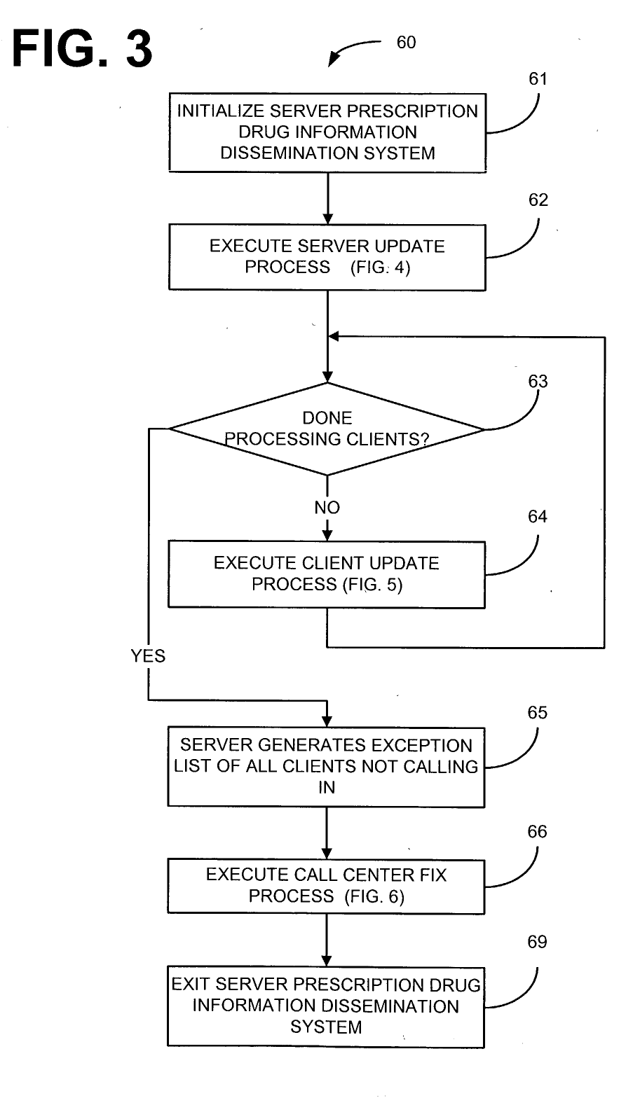

Illustrated in FIG. 3 is a flow chart depicting an example of the process flow of the server prescription drug information dissemination system 60 of the present invention as shown in FIG. 2A. The server prescription drug information dissemination system acquires updates to the prescription drug information database and processes the update data into components that can be accessed on an as needed basis by the client 16.

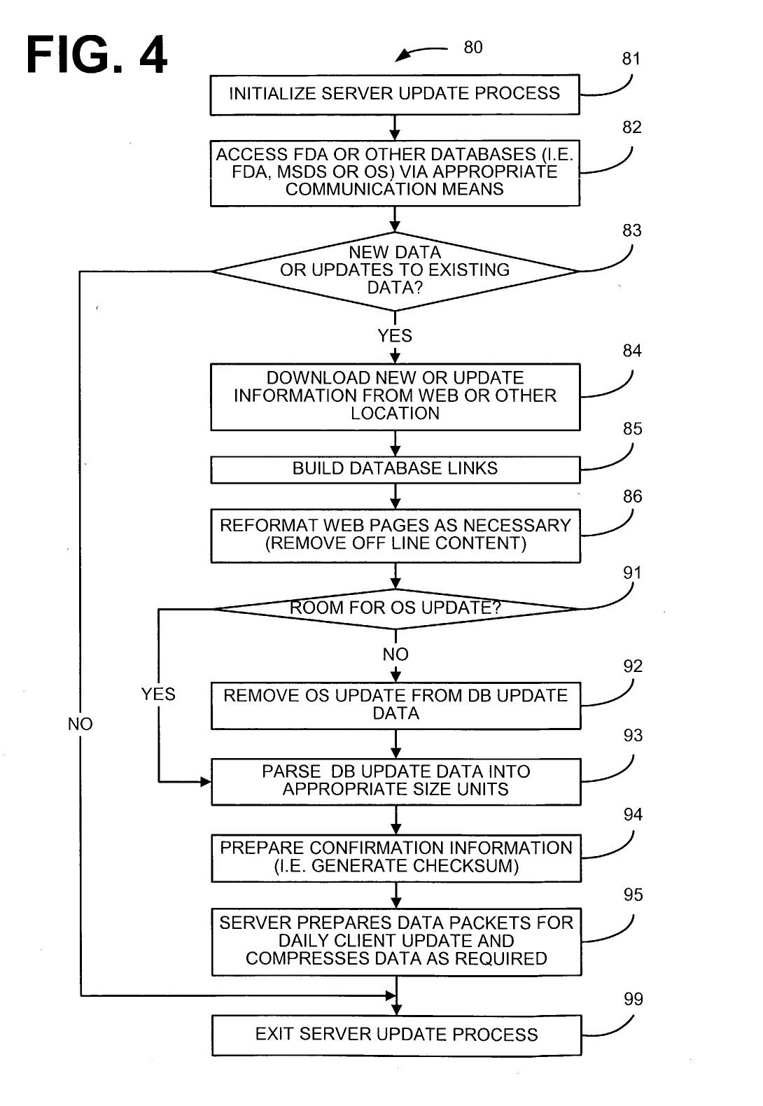

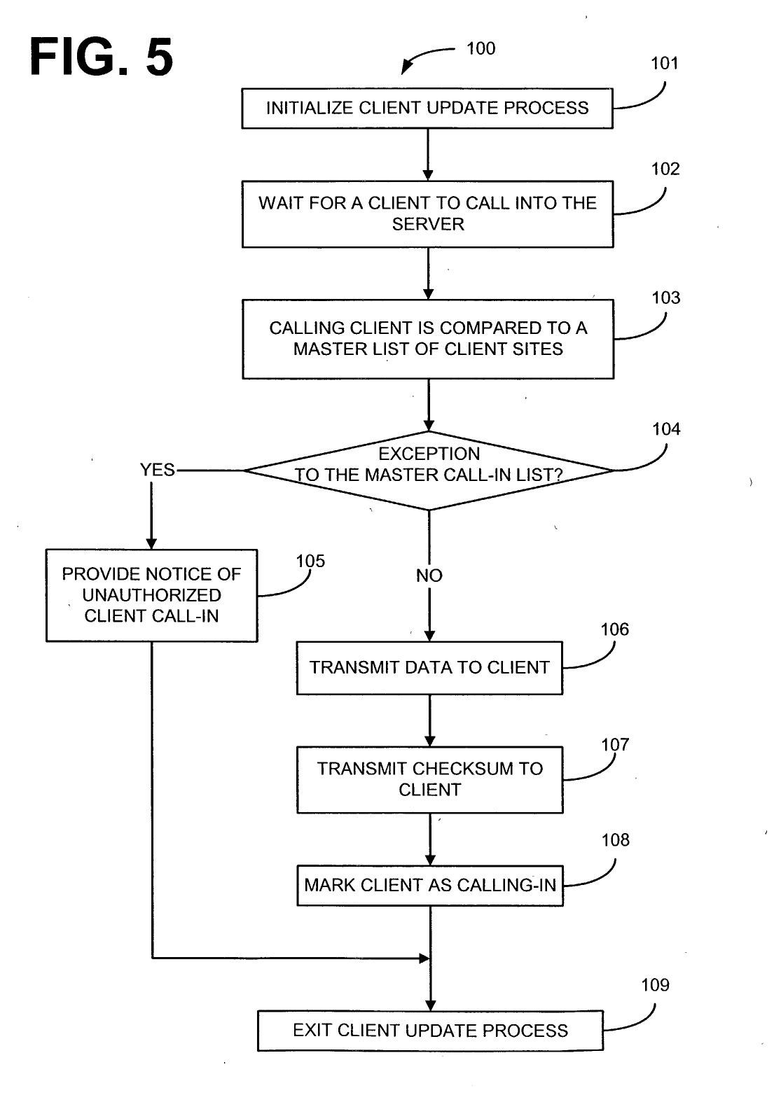

First at step 61, the server prescription drug information dissemination system is initialized. At step 62, the server update process is performed. The server update process is herein defined in further detail with regard to FIG. 4. After performing the server update process at step 62, the server prescription drug information dissemination system 60 then determines if it is done processing clients at step 63. If it is determined at step 63 that there are more clients to be processed, the server prescription drug information dissemination system 60 executes the client update process at step 64. The client update process is herein defined in further detail with regard to FIG. 5. After performing the client update process at step 64, the server prescription drug information dissemination system 60 then returns to determine if there are more clients requiring process at step 63.

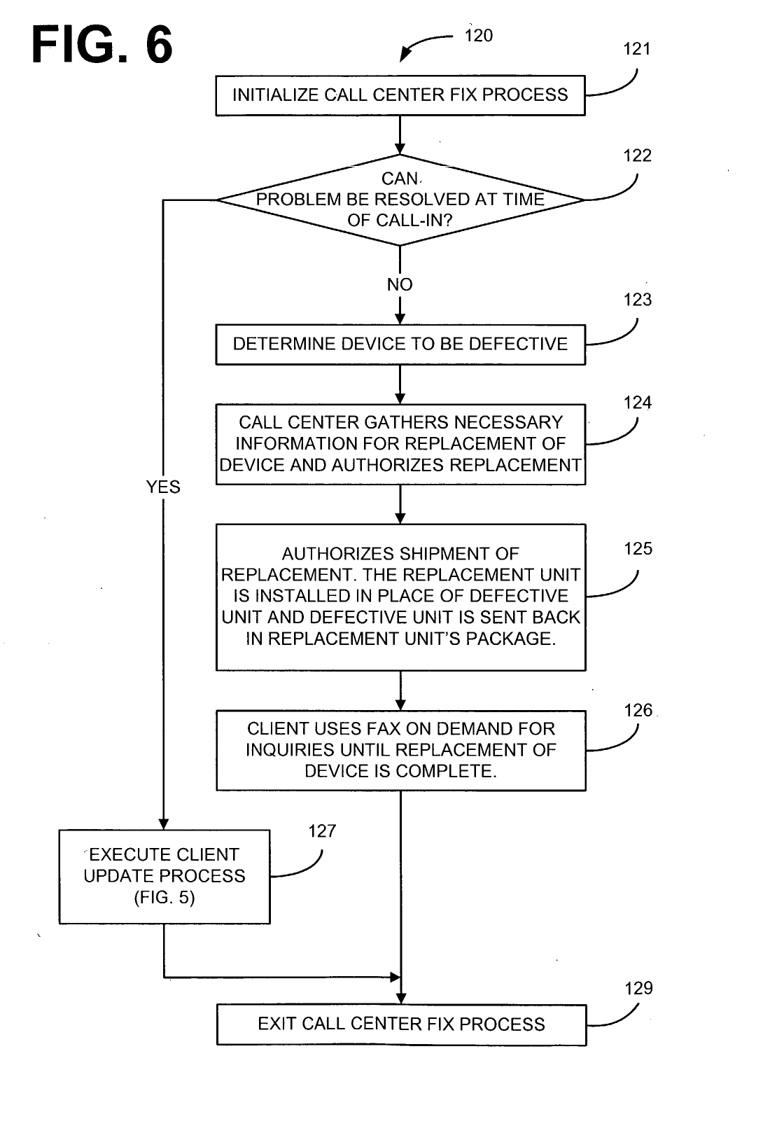

However, if it is determined at step 63 that there are no more clients to be processed then the server prescription drug information dissemination system 60 generates an exception list of all clients not calling in at step 65. At step 66, the call center fix process is executed. The call center fix process is herein defined in further detail with regard to FIG. 6. After performing the call center fix process at step 66, the server prescription drug information dissemination system then exits at step 69.

Illustrated in FIG. 4 is a flow chart illustrating an example of the server update process 60, as shown in FIGS. 2A and 3, operating with the server prescription drug information dissemination system 60 of the present invention. The server update process 80 acquires updated information from a variety of sources such as, but not limited to, the FDA, MSDS or operating system. This update information is used to construct a database update data which is prepared for dissemination to the client 16.

First the server update process 80 is initialized at step 81. At step 82, the server update process 80 then accesses the appropriate databases to determine if there are updates to the predetermined list of databases. The predetermined list of databases includes, but is not limited to, the FDA, prescription drug databases, MSDS, or, O/S operating system databases or the like. After accessing the predetermined databases, the server update process then determines if there is new data or updates to existing data at step 83. If it is determined at step 83 that there is no new data or updates to the existing data, then the server process 80 proceeds to step 99 and exits.

However, if it is determined at step 83 that there is new data or updates to existing data, then the server update process 80 downloads the new or update information from the predetermined database sources. These predetermined database sources can be accessed from the web or other locations. Other locations include other network access to the predetermined databases. At step 85 the server update process 80 then builds the database links required for constructing the database update data. At step 86, the server update process 80 reformats web pages as necessary and removes any offline content. At step 91, the server update process 80 determines if there is room for any O/S update data. If it is determined at step 91 that there is room for the operating system update data, then the server update process 80 proceeds to step 93. However, if it is determined at step 91 that there is no room for operating system updates, then the server update process 80 removes the operating system update from the database update data at step 92.

At step 93, the server update process 80 parses the database updated data into appropriate size units for dissemination. At step 94, the confirmation information is prepared. This confirmation information includes, but is not limited to, checksums and the like. At step 95, the server update process 80 prepares data packets for the daily client update and compresses the data as required. At step 99, the server update process exits.

Illustrated in FIG. 5 is a flow chart illustrating an example of the client update process 100 as shown in FIGS. 2A and 3, operating within the server prescription drug information dissemination system 60 of the present invention. The client update process 100 is responsible for the actual transmission of data to the clients 16.

First, the client update process 100 is initialized at step 101. At step 102, the client update process waits for clients to call in to the server. At step 103, the calling clients are compared to the master list of client sites and the client update process 100 notes if the call-in process is successful. If it is determined at step 104 that the call-in client is an exception to the master call-in list, then the client update process 100 provides a notification of an unauthorized client attempting to call in at step 105. This notice is intended to prompt an investigation into the identity of the unauthorized client that is not listed on the master call-in list, and to provide information so the appropriate action may be taken. After performing the notification the client update process 100 then exits at step 109.

However, if the call-in process is successful and it is determined at step 104 that the call-in client is not an exception to the master call-in list, then the client update process 100 transmits the database update data to the client at step 106 and transmits the checksum to the client at step 107. At step 108, the client update process marks the call-in client as having called in and then exits at step 109.

Illustrated in FIG. 6 is a flow chart illustrating an example of the call center fix process 120, as shown in FIGS. 2A, 3 and 5, operating in the server prescription drug information dissemination system 60 of the present invention. Call center fix process 120 enables help processes at the server site to diagnose problems with clients connecting to the server 11.

First, the call center fix process 120 is initialized at step 101. At step 122, the call center fix process then determines if the problem can be resolved at the time of the call-in. If it is determined at step 122 that the problem can be resolved at the time of the call-in, then the call center fix process 120 then proceeds to step 127 to execute the client update process. The client update process was herein defined previously with regard to FIG. 5. After performing the client update process, the call fix process then exits at step 129.

However, if it is determined at step, 122 that the problem could not be resolved at the time of the call-in, then the call center fix process 120 determines that the device is defective at step 123. At step 124, the call center gathers the necessary information for replacement of the device determined to be defective and authorizes the replacement. At step 125, the authorization of the shipment of the replacement device is performed. When the replacement device is received at the client site, the replacement unit is installed in place of the defective unit and the defective unit is sent back in the replacement unit's package. At step 126, the client is requested to use the fax on demand system for inquiries until replacement of the device is completed. The call center fix process 120 then exits at step 129.

Illustrated in FIG. 7 is a flowchart illustrating an example of the client prescription drug information dissemination system 140 as shown in FIGS. 2B and 3. operating on the client 11 in conjunction with the server prescription drug information dissemination system 60 (FIGS. 2A, 3). The client prescription drug information dissemination system 140 resides on the client device 16 and performs the updates to the mirror prescription drug information database 17. The client prescription drug information drug dissemination system 140 also enables a user to access the information within the mirror prescription drug information database utilizing a query system.

First, the client prescription drug information dissemination system 140 is initialized. At step 142, the client prescription drug dissemination system 140 determines if an update process is to be performed. If it is determined at step 142 that it is not time for the update process to be performed then the client prescription drug information dissemination system 140 then proceeds to step 151. However, if it is determined at step 142 it is time for the update process to be performed, then the client prescription drug information dissemination system 140 executes the update process at step 143. The update process is herein defined in further detail with regard to FIG. 8.

At step 144, it is determined whether the update occurring at step 143 was successful. If it is determined at step 144 that the update occurring at step 143 was not successful, then the client prescription drug information dissemination system 140 informs the client to use the fax on demand for queries until the call center has corrected the problem with the updates at step 145. The client prescription drug information dissemination system 140 then proceeds to step 151. However, if it is determined at step 144 that the update occurring at step 143 was successful, then the client prescription drug information dissemination system 140 determines if there are any alerts present at step 146.

If it is determined at step 146 that there are no alerts present, then the client prescription drug information dissemination system 140 then proceeds to step 151. However, if is determined at step 146 that there are alerts present, then the alerts are processed at step 147. These alerts are notifications of conditions that the client 11 is to be aware of for complete notification. The alert information includes, but is not limited to, notifying a user of the selected databases related to a visual alert. The alert process includes enabling a user to select the database related to the visual alert. After selecting the database related to the visual alert, the client prescription drug information dissemination system 140 displays a list of alerts for the selected database. The user is then able to select the most recent alert so that the client prescription drug information dissemination system can display the text of that alert. Also in the processing of the alert information, the client prescription drug information dissemination system 140 then keeps a log reporting which alerts were reviewed by the user.

At step 151, the client prescription drug information dissemination system 140 then determines if the user is requesting to perform a query. If it is determined at step 151 that the user is not requesting to perform a query, then the client prescription drug information dissemination system 140 then proceeds to step 154. However, if it is determined at step 151 that the user has requested to perform a query, then the query process is executed at step 152. The query process is herein defined in further detail with regard to FIG. 10. After performing the requested query, the client prescription drug information dissemination system 140 then determines if the user has requested more queries to be processed at step 153. If it is determined at step 153 that there are more queries to be processed then the client prescription drug information dissemination system 140 returns to repeat steps 152 and 153.

At step 154, the client prescription information dissemination system 140 then determines if it is done processing data on the client 11. If client prescription drug information dissemination system 140 then determines that there is further processing to be performed then the client prescription drug information dissemination system 140 then returns to repeat steps 142-154. However, if it is determined at step 154 that there are no more processes to be performed then the client prescription drug information dissemination system 140 exits at step 159.

Illustrated in FIG. 8 is a flowchart illustrating an example of the update process 160 as shown in FIGS. 2A and 7, operating with the client prescription drug information dissemination system 140. The update process 160 performs the steps required to access, download and update the prescription drug database 16 (FIG. 2A).

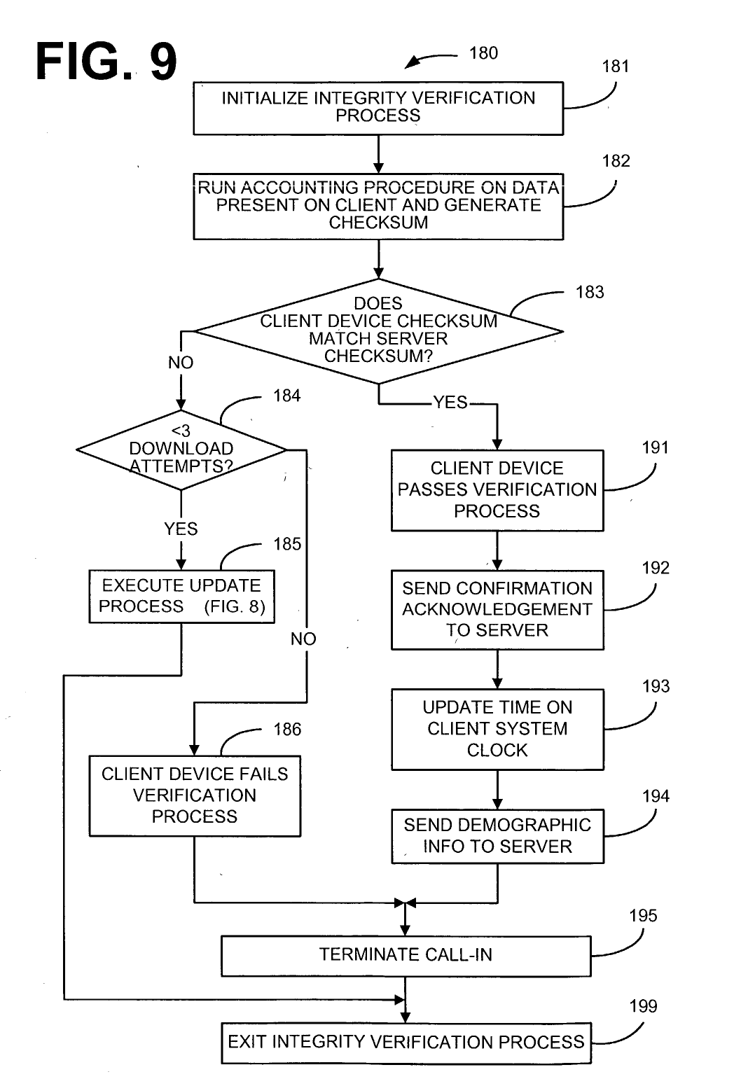

First the update process 160 is initialized at step 161. At step 162, the update process 160 accesses the server 11 (FIGS. 1 and 2A) via the appropriate communication means. The appropriate communication means includes, but is not limited to, communication through network 14 via the internet, LAN, WAN, PSTN, cable modem or the like. At step 163, the update process 160 downloads any new or updated data from the server and checksum from the server at step 164. At step 165, the update process 160 decompresses any data needed on the client. At step 166, the update process 160 updates the mirror prescription drug information database 17 (FIGS. 1, 2B) at step 166. At step 167, the update process 160 performs the integrity verification process. The integrity verification process herein defined in further with regard to FIG. 9. The update process 160 then exits at step 169.

Illustrated in FIG. 9 is a flowchart depicting an example of the integrity verification process 180, as shown in FIG. 2B and FIG. 8, operating process 160 as shown in FIG. 2B and FIG. 8. The integrity verification process 180 determines the integrity of the database after applying the database update data to the mirror prescription drug information database 17 (FIGS. 1, 2B).

First the integrity verification process 180 is initialized at step 181. At step 182 the integrity verification process then runs an accounting procedure on the data present on the client and generates a client checksum. At step 183, the integrity verification process 180 determines if the client device checksum matches the server checksum at step 164 (FIG. 8). If it is determined at step 183 that the checksum generated on the client device does not match the checksum received from the server, then the integrity verification process 180 proceeds to step 184. At step 184, the integrity verification process 180 determines if the update process 160 has attempted more than three downloads of the database updated data. If it is determined at step 184 that there have been less than three attempts by the update process 160 (FIG. 8) to download data, then the integrity verification 180 then executes the update process at step 185. The update process was herein defined in further detail with regard to FIG. 8. After performing the update process, the integrity verification process 180 then exits at step 199.

However, if it is determined at step 184 that the update process 160 has attempted three downloads of new or updated data from the server, then the integrity verification process 180 indicates that the client device fails the verification process at step 186. After marking that the client device is failing the verification at step 186, the integrity verification process 180 then terminates the call at step 195 and exits at step 199.

In the preferred embodiment, the integrity verification process 180 does not change the update time when the update process has failed verification. In alternative embodiments, the integrity verification process 180 will indicate to a user that the client device has failed the verification process at step 186. These indications to the user that the client device has failed the verification process include, but are not limited to, warning lights, error messages, printed line messages, error tones and the like.

However, if it is determined at step 183 that the client device checksum does match the checksum receipt from the server, then the integrity verification process 180 indicates that the client device has passed the verification process at step 191. At step 192, the integrity verification process 180 then sends a confirmation acknowledgement to the server. At step 193, the integrity verification process 180 then updates the time on the client system clock to synchronize it with the time on the server system clock to facilitate an accurate indication of the time that the client device was last successfully updated. At step 194, the integrity verification process 180 then sends demographic information to the server. This demographic information includes identification information on the client as well as the time from the client system clock indicating the time at which the client device was successfully updated. At step 195, the integrity verification process 180 then terminates the call-in and exits at step 199.

Illustrated in FIG. 10 is a flowchart depicting an example of the query process 200, as shown in FIG. 2B and FIG. 7, operating with the client prescription drug information dissemination system 140 of the present invention. The query process 200 enables a user to access the information in the mirror prescription drug information database 17 (FIGS. 1 and 2B).

First the query process 200 is initialized at step 201. At step 202, the query process 200 enables a user to select the type of data to be accessed. At step 203, the query process enables a user to select the type of query to be run. Examples of the type of queries to be run include, but are not limited to, searches with regard to brand name, generic name, NDC numbers and the like. After enabling the user to select the type of query to be run at step 203, the query process 200 then executes the query on the data selected at step 204.

The output of results for the query matches are performed at step 205. The output result of the query matches may include, but are not limited to, text outputs to a screen, data output to a printer 18 (FIG. 1) or other type of output device. After outputting the results for the query matches at step 205, the query process 200 then determines if the user wishes to further refine the search at step 206. If it is determined at step 206 that the user does wish to refine a search, the query process 200 then enables the user to modify the query with new or changed query terms at step 207. The query process 200 then returns to repeat steps 204 through 206.

However, if it is determined at step 206 that the user does not need a further refinement of the search, then the query process 200 determines if the query is to be printed at step 211. If it is determined at step 211 that the query is not to be printed, then the query process 200 then skips to step 213. However, if it is determined at step 211 that the query is to be printed, then the query is printed at step 212. At step 213, the query process 200 determines if the user wishes to run additional queries. If it is determined at step 213 that the user does wish to run additional queries, then the query process 200 returns to repeat steps 202-213. However, if it is determined at step 213 that the user does not wish to run additional queries, then the query process 200 exits at step 219.

It will be apparent to those skilled in the art that many modifications and variations may be made to embodiments of the present invention, as set forth above, without departing substantially from the principles of the present invention. All such modifications and variations are intended to be included herein within the scope of the present invention, as defined in the claims that follow.

What is claimed is:

1. A method for disseminating drug information on a server to a remote device, comprising the steps of:
creating update drug data for addition to original drug information;
waiting for the remote device to request update drug data; and
transmitting the update drug data to the remote device.

2. The method of claim 1, wherein said step of creating the update drug data further comprises the step of:
accessing remote data sources to check for updated drug information;
accumulating the updated drug information into the update drug data; and
compressing the update drug data if required.

3. The method of claim 1, wherein said drug information includes prescription drug package insert information.

4. The method of claim 1, further comprising the step of:
verifying the remote device is authorized to receive the update drug data.

5. The method of claim 4, further comprising the step of:
logging all remote devices verified as authorized to receive the update drug data; and
generating an exception list of remote devices authorized to receive the update drug data and not successfully receiving the update drug data in a predetermined time period.

6. A system for disseminating drug information, comprising:
a server device containing original drug information, wherein the server device further comprises:
a update mechanism that creates update drug data for addition to the original drug information;
a transmit mechanism that transmits the update drug data to the remote device upon receiving a request from a remote device for the update drug data.

7. The system of claim 6, wherein the update mechanism further comprises:
a data check mechanism to check for updated drug information;
a data build mechanism that accumulates the updated drug information into the update drug data; and
a data compression mechanism that compresses the update drug data if required.

8. The system of claim 6, wherein said drug information includes prescription drug package insert information.

9. The system of claim 6, further comprising:
a verification mechanism that verifies if the remote device is authorized to receive the update drug data.

10. The system of claim 9, wherein the verification mechanism further comprises:
a log mechanism that logs all remote devices verified as authorized to receive the update drug data; and
a list generation mechanism that generates an exception list of remote devices authorized to receive the update drug data and not successfully receiving the update drug data in a predetermined time period.

11. A computer readable medium for a logic that disseminates drug information, comprising:
logic for creating update drug data for addition to original drug information;
logic for waiting for a remote device to request update drug data; and
logic for transmitting the update drug data to the remote device.

12. The computer readable medium of claim 11, wherein the logic for creating further comprises:
logic for accessing remote data sources to check for updated drug information;
logic for accumulating the updated drug information into the update drug data; and
logic for compressing the update drug data if required.

13. The computer readable medium of claim 11, wherein said drug information includes prescription drug package insert information.

14. The computer readable medium of claim 11, further comprising
logic for verifying the remote device is authorized to receive the update drug data.

15. The computer readable medium of claim 14, wherein the logic for verifying further comprises:
logic for logging all remote devices verified as authorized to receive the update drug data; and
logic for generating an exception list of remote devices authorized to receive the update drug data and not successfully receiving the update drug data in a predetermined time period.

16. A system for disseminating drug information on a server to a remote device, comprising:
means for creating update drug data for addition to original drug information;
means for waiting for a remote device to request update drug data; and
means for transmitting the update drug data to the remote device.

17. The system of claim 16, further comprising:
means for accessing remote data sources to check for updated drug information;
means for accumulating the updated drug information into the update drug data; and
means for compressing the update drug data if required.

18. The system of claim 16, wherein said drug information includes prescription drug package insert information.

19. The system of claim 16, further comprising:
means for verifying the remote device is authorized to receive the update drug data.

20. The system of claim 19, further comprising:
means for logging all remote devices verified as authorized to receive the update drug data; and
means for generating an exception list of remote devices authorized to receive the update drug data and not successfully receiving the update drug data in a predetermined time period.
